(19) United States
(12) Patent Application Publication
(10) Pub. No.: US 2025/0321229 A1
(43) Pub. Date: Oct. 16, 2025

(54) NEW MARKER FOR PROSTATE CANCER

(71) Applicant: NATIONAL UNIVERSITY CORPORATION KUMAMOTO UNIVERSITY, Kumamoto (JP)

(72) Inventors: Norie ARAKI, Kumamoto (JP); Akira SASAO, Kumamoto (JP)

(73) Assignee: NATIONAL UNIVERSITY CORPORATION KUMAMOTO UNIVERSITY, Kumamoto (JP)

(21) Appl. No.: 18/708,283

(22) PCT Filed: Nov. 14, 2022
(86) PCT No.: PCT/JP2022/042221

§ 371 Date: Jan. 29, 2025

(30) Foreign Application Priority Data
Nov. 12, 2021 (JP) 2021-184569

(51) Int. Cl. G01N 33/574

(52) U.S. Cl. CPC G01N 33/57434; G01N 33/57488; G01N 2440/00

(57) ABSTRACT

An object of the present invention is to provide a new marker for prostate cancer and a method for detecting prostate cancer using the marker. The present invention provides a method for determining prostate cancer comprising the following steps of: (a) releasing PSA from a PSA-ACT complex present in a sample derived from a subject; (b) performing two-dimensional electrophoresis on the free PSA obtained in the step (a) to separate PSA and detecting a PSA spot; and (c) verifying the presence of the PSA spot detected on the basic side compared to the control PSA spot.

### TECHNICAL FIELD

The present invention relates to a new marker for prostate cancer. The present invention also relates to a method for detecting prostate cancer using the marker.

### BACKGROUND ART

In Japan, the number of deaths from prostate cancer is 5% of all male cancer deaths, while the number of cases is approximately 10% of all male cancers, and the rate of increase in deaths in recent years is the highest among all cancers. Prostate-specific antigen (PSA) testing is widely used in the early diagnosis and treatment prognosis prediction of prostate cancer, but although the testing method is highly sensitive, it has low specificity, and it is difficult to distinguish between biological malignancies. Therefore, false positives are likely to occur, and detailed tests such as ultrasound tests and rectal examination are required. Furthermore, definitive diagnosis of prostate cancer mainly requires invasive tests such as biopsies, which places a heavy burden on patients. Frequent false positives result in unnecessary medical procedures and burden on patients, resulting in great stress for patients and economic loss. Furthermore, treatments after definitive diagnosis mainly involve surgical excision, radiation therapy, hormone therapy, etc., but diagnostic markers for prognosis and sensitivity of them do not exist other than PSA. Therefore, it is essential to construct a diagnostic system with low invasiveness and high specificity for early diagnosis and determination of treatment effectiveness, and immediate improvement measures are required in terms of patient pain and economic efficiency.

Since a 2010 British meta-analysis study showed that PSA testing intervention reduced mortality rates in the medical examination group, the Japanese Urological Association also recommends this. Takakura et al. have reported a new tumor marker candidate through an investigation using a liquid chromatography mass spectrometer (Non-Patent Document 1: ISRN Oncol 2012, 768190). Furthermore, according to a report by Jung et al. (Non-Patent Document 2: ClinChem 2004, 50:2292-2301), it is suggested that the degree of malignancy may be possibly differentiated by the fractionation pattern of PSA in two-dimensional electrophoresis. However, there have been no reports regarding the differentiation between those sensitive to hormone therapy and those that are castration-resistant, among malignant tumors. Furthermore, according to a report by Gloria et al. (Non-Patent Document 3: Glycobiology 2006, 16:132-145), it has been shown that PSA exhibits various patterns when subjected to two-dimensional electrophoresis. Furthermore, it has been reported that it may be possible to distinguish between benign and malignant lesions based on this pattern difference. However, there is no report that they could be clearly distinguished. As described above, although previous papers have attempted to distinguish between prostatic hyperplasia or prostatitis and prostate cancer, there have been no reports that differentiate castration resistance. Furthermore, with these methods, it is difficult to verify that a patient with prostatic hyperplasia or prostatitis does not substantially have prostate cancer.

It is known that PSA is a glycoprotein with a molecular weight of approximately 30,000 and has asparagine-linked (N-type) sugar chains, and the terminal of the sugar chain is sialylated. Furthermore, PSA derived from prostate cancer patients is reported to have a LacdiNAc structure, which is a disaccharide in which N-acetylgalactosamine and N-acetylglucosamine are bonded.

### PRIOR ART DOCUMENT

### Non-Patent Document

[Non-Patent Document 1] Takakura et al., ISRN Oncol 2012, 768190
[Non-Patent Document 2] Jung et al., Clin Chem 2004, 50:2292-2301
[Non-Patent Document 3] Gloria et al., Glycobiology 2006, 16:132-14

### SUMMARY OF THE INVENTION

### Technical Problem

An object of the present invention is to provide a method for detecting prostate cancer using a new marker for prostate cancer.

### Solution to Problem

The present inventors have intensively studied to solve the above problems, and resultantly found that PSA having specific properties exists in the blood of prostate cancer patients, leading to completion of the present invention.

The present invention includes the following.
[1]A method for determining that a subject is suffering from or is suspected of suffering from prostate cancer by detecting prostate-specific antigen (PSA) present in a sample derived from the subject, characterized in that when the isoelectric point(s) of PSA(s) derived from the subject is on the basic side compared to PSA(s) derived from a healthy person, it is determined that the subject is suffering from or is suspected of suffering from prostate cancer.
[2] The determination method according to [1] above, wherein when the isoelectric point of at least a portion of the PSA(s) derived from the subject is more basic than the isoelectric point of any PSA derived from a healthy person, when the fraction of PSA(s) that has basic isoelectric point derived from the subject is larger than the fraction of PSA(s) that has basic isoelectric point derived from a healthy person, when the distribution of the isoelectric point(s) of the PSA(s) derived from the subject is on the basic side compared to the distribution of the isoelectric point(s) of the PSA(s) derived from a healthy person, or when a PSA with an isoelectric point of 6.0 or higher is detected, then it is determined that the isoelectric point(s) of the PSA(s) derived from the subject is on the basic side compared to PSA(s) derived from a healthy person.
[3] The determination method according to [1] above, wherein when the fraction of PSA(s) that has basic isoelectric point derived from the subject is larger than the fraction of PSA(s) that has basic isoelectric point derived from a healthy person, or when the distribution of the isoelectric point(s) of PSA(s) derived from the subject is on the basic side compared to the distribution of the isoelectric point(s) of PSA(s) derived from a healthy person, then it is determined that the isoelectric point(s) of the PSA(s) derived from the subject is on the basic side compared to PSA(s) derived from a healthy person.
[4] The method according to any one of [1] to [3] above, wherein the sample is serum derived from the subject.
[5] The method according to any one of [1] to [4] above, wherein the sample is serum derived from the subject treated with ethanolamine.
[6] The method according to any one of [1] to [5] above, wherein the subject is further determined to have castration-resistant prostate cancer based on the degree of basicity of the isoelectric point of PSA derived from the subject.
[7] The method according to any one of [1] to [5] above, wherein when PSA with an isoelectric point of 6.5 or more is detected, the subject is further determined to have castration-resistant prostate cancer.
[8]A method for determining that a subject is suffering from or is suspected of suffering from prostate cancer by detecting prostate-specific antigen (PSA) present in a sample derived from the subject, wherein when PSA(s) derived from the subject is(are) desialylated compared to PSA(s) derived from a healthy person, it is determined that the subject is suffering from or is suspected of suffering from prostate cancer.
[9] The method according to [8] above, wherein the sample derived from the subject is serum derived from the subject.
[10] The method according to [8] above, wherein the sample derived from the subject is a subject-derived serum treated with ethanolamine.
[11] The method according to any one of [8] to [10] above, wherein the subject is further determined to have castration-resistant prostate cancer based on the degree of desialylation of PSA derived from the subject.
[12]A method for obtaining information used to diagnose that a subject patient is suffering from or is suspected of suffering from prostate cancer, wherein the method comprises detecting prostate-specific antigen (PSA) present in serum derived from the patients suspected of having prostate cancer, and determining whether the isoelectric point(s) of the detected PSA(s) derived from the subject patient is on the basic side compared to PSA(s) derived from a healthy person.
[13] The method according to [12] above, wherein when the isoelectric point(s) of at least a portion of PSA(s) derived from the subject is more basic than the isoelectric point of any PSA derived from a healthy person, when the fraction of PSA(s) that has basic isoelectric point derived from the subject is larger than the fraction of PSA(s) that has basic isoelectric point derived from a healthy person, when the distribution of the isoelectric point(s) of PSA(s) derived from the subject is on the basic side compared to the distribution of the isoelectric point(s) of PSA(s) derived from a healthy person, or when a PSA with an isoelectric point of 6.0 or higher is detected, then it is determined that the isoelectric point(s) of PSA(s) derived from the subject is on the basic side compared to PSA(s) derived from a healthy person.
[14] The method according to [12] or [13] above, wherein the serum derived from the patient is a patient-derived serum treated with ethanolamine.
[15]A method for obtaining information used to diagnose that a subject patient is suffering from or is suspected of suffering from prostate cancer, wherein the method comprises detecting prostate-specific antigen (PSA) present in serum derived from the patient suspected of having prostate cancer, and determining whether the detected PSA(s) is(are) desialylated compared to PSA(s) derived from a healthy person.
[16] The method according to [15] above, wherein the serum derived from the patient is a patient-derived serum treated with ethanolamine.
[17]A method for detecting prostate-specific antigen (PSA) present in a sample derived from a subject, comprising the following steps of:
(a) releasing PSA from a PSA-ACT complex present in the sample derived from the subject;
(b) performing two-dimensional electrophoresis on the free PSA obtained in the step (a) to separate PSAs with different isoelectric points and detecting each PSA spot; and
(c) verifying the presence of the PSA spot(s) detected on the basic side compared to the control PSA spot (s).
[18] The method according to [17] above, wherein the sample derived from the subject is a human-derived serum.
[19] The method according to [17] or [18] above, further comprises performing albumin/IgG removal treatment on serum before the step (a).
[20] The method according to any one of [17] to [19] above, wherein the control PSA is a PSA derived from a healthy human.
[21] The method according to any one of [17] to [20] above, wherein the step (a) is carried out using ethanolamine.
[22] The method according to any one of [17] to [21] above, when a PSA spot is detected on the basic side in the step (c), it is determined that the subject is suffering from prostate cancer.
[23] The method according to [22] above, wherein it is determined whether the subject is further suffering from castration-resistant prostate cancer, depending on the extent of the PSA spot(s) detected on the basic side compared to the control PSA spot, in the step (c).
[24]A method for diagnosing that a subject is suffering from prostate cancer or castration-resistant prostate cancer, based on the determination by the method described in any one of [1] to [16] above.
[25]A method for diagnosing whether or not the prostate cancer the subject is suffering from is castration-resistant prostate cancer, and deciding a treatment policy, based on the determination by the method described in any one of [1] to [16] above.
[26] The method according to [25] above, wherein the treatment policy is a decision on whether to perform hormone therapy or chemotherapy.
[27]A method for evaluating the effectiveness of medication by detecting prostate-specific antigen (PSA) present in serum derived from prostate cancer patients receiving medication and comparing the detected PSA before and after medication, wherein the method comprises checking (a) a decrease in the amount of PSA with a basic isoelectric point, or (b) a decrease in the amount of desialylated PSA, before and after medication.
[28] The method according to [27] above, wherein the comparison of PSA before and after medication is performed using two-dimensional electrophoresis.
[29] The method according to [27] or [28] above, which determines whether or not medication is effective based on the comparison of PSA before and after medication.

### Advantageous Effect of the Invention

The present invention provides a new marker for prostate cancer and a method for detecting prostate cancer using the marker.

### BRIEF DESCRIPTION OF DRAWINGS

FIG. 1 shows the results of two-dimensional electrophoresis of seminal plasma derived from a healthy person. The results of protein staining are shown. The circled spots indicate nine representative spots (0-8) that were positive for an anti-PSA antibody.

FIG. 2 shows the results of two-dimensional electrophoresis of a cell lysate of prostate cancer cell LNCaP. The upper left figure shows the results of protein staining. The lower left figure shows the results of staining with an anti-PSA antibody. The lower right figure shows an enlarged view of the boxed area in the lower left figure. Spots that were positive for an anti-PSA antibody are circled. The upper right figure is a three-dimensional image of the eight PSA-positive spots circled in the lower right figure.

FIG. 3 shows the results of two-dimensional electrophoresis of various samples (seminal plasma derived from a healthy person, cell lysate of LNCaP cell, cell lysate of C4-2 cell, and PC-3 cell lysate). The left figure shows the results of staining with an anti-PSA antibody. An enlarged view of the location where PSA positive spots were detected is shown. The right figure is a three-dimensional image of each PSA-positive spot.

FIG. 4 shows the results of two-dimensional electrophoresis of serum derived from prostate cancer patients. Serum derived from hormone-sensitive prostate cancer patients (serum A) and serum derived from castration-resistant prostate cancer patients (serum B and C) were used. As a control, seminal plasma derived from a healthy person was used. The figures show the results of staining with an anti-PSA antibody, and show enlarged views of the location where PSA-positive spots were detected.

FIG. 5 shows three-dimensional images (left) of PSA-positive spots in the prostate cancer patient serum results of FIG. 4 and comparisons of the detected spot intensities (right figures).

FIG. 6 shows the results of verifying the isoelectric point shift of PSA when serum from castration-resistant prostate cancer patients was treated with sialidase. The left figures show the results of staining with an anti-PSA antibody, and show enlarged views of the location where PSA-positive spots were detected. The middle figures show three-dimensional images of PSA-positive spots (left), and the right figures show comparisons of the detected spot intensities.

FIG. 7 shows the results of a comparison of the intensity of PSA-positive spots using serum derived from hormone-sensitive prostate cancer patients and serum derived from castration-resistant prostate cancer patients.

### DESCRIPTION OF EMBODIMENTS

The invention is described below by way of exemplary embodiments, along with preferred methods and materials that can be used in the practice of the invention. It should be noted that, unless otherwise specified herein, all technical and scientific terms used herein have the same meanings as commonly understood by those skilled in the art to which this invention pertains. Also, any materials and methods equivalent or similar to those described herein can also be used in the practice of the invention. Additionally, all publications and patents cited in the present specification in connection with the invention described in the present specification constitute a part of the present specification as indicating, for example, methods, materials, etc., that can be used in the present invention.

In the present specification, when the expression “X to Y” is used, it is used to mean that the lower limit is X and the upper limit is Y, or the upper limit is X and the lower limit is Y.

The present inventors have successfully developed a fully automatic two-dimensional electrophoresis device (Auto 2D) as a highly sensitive and rapid quantitative pathological protein analysis device. Detection of pathological markers by 2D-WB (two-dimensional electrophoresis-Western blotting) has until now required a huge amount of time, cost, and technology, making it difficult to introduce into medical examinations or regular medical treatment. Using Auto 2D, it has become possible to detect pathological markers using 2D-WB with an amount of 1/100 or less, with high sensitivity and high precision, and fully automatically, within 1 to 2 hours.

The present inventors have attempted to develop a system that can accurately and quickly analyze the distribution and amount of prostate cancer marker proteins, including post-translational modifications, and discovered that 32 types or more of post-translational modification (sugar chain, phosphorylation, oxidation, aggregate, protein decomposition product, etc.) spots of prostate cancer-specific PSA can be detected (all identified by mass spectrometry) with good reproducibility on a 2D chip, from serum, prostatic fluid, and prostate cancer cells. In addition, it has also been discovered that these spots exhibit a specific profile that allows them to distinguish between normal and cancers, as well as whether they are sensitive to AR inhibitors or anticancer drugs such as taxol, or to hormone therapy.

One aspect of the present invention is a method for determining that a subject is suffering from or is suspected of suffering from prostate cancer (hereinafter, the term “suffering from prostate cancer” will be collectively adopted unless it is clear from the context that only one is being referred to) by detecting prostate-specific antigen (PSA) present in a sample derived from the subject to be determined, wherein when the isoelectric point of the detected PSA is on the basic compared to PSA present in prostatic fluid derived from a healthy person (hereinafter, sometimes referred to as “normal PSA”), it is determined that the subject is suffering from prostate cancer. Here, the case wherein the isoelectric point is on the basic compared to normal PSA includes, for example, (1) a case when the isoelectric point of at least a portion of the PSA derived from the subject is the basic compared to the PSA derived from a healthy person (preferably, PSA derived from a healthy person, which is the most basic), (2) a case when the fraction in which the isoelectric point of PSA derived from the subject is basic is greater than the fraction in which the isoelectric point of PSA derived from a healthy person is basic, (3) a case when the distribution of the isoelectric point of PSA derived from the subject is on the basic side compared to the distribution of the isoelectric point of PSA derived from a healthy person, and (4) a case when a PSA with an isoelectric point of 6.0 or higher is detected.

Another aspect of the present invention is a method for detecting prostate-specific antigen (PSA) present in a sample derived from a subject, comprising the following steps of: (a) releasing PSA from a PSA-ACT (PSA-al antichymotrypsin) complex present in the sample derived from the subject; (b) performing two-dimensional electrophoresis on the free PSA present in the sample (free PSA originally existing in free form and free PSA obtained in step (a)) to separate PSAs based on the isoelectric point and molecular weight, and detecting each PSA spot; and (c) verifying the presence of the PSA spot(s) detected on the basic side compared to the control PSA spot(s). Here, the control PSA is preferably PSA present in prostatic fluid derived from a healthy person.

Another aspect of the present invention is a method for determining that the subject is suffering from prostate cancer, further comprising a step (d) wherein when a PSA spot is detected on the basic side in the step (c), it is determined that the subject is suffering from prostate cancer.

Another aspect of the present invention is a method comprising determining that the subject is suffering from castration-resistant prostate cancer, depending on the degree of basicity of the PSA spot detected on the basic side, in the step (d).

Each will be explained below.

The subject-derived sample used in the method of the present invention is a body fluid derived from a mammal, preferably a human. The body fluid is not particularly limited as long as PSA can be detected therein, but urine or blood is preferred, and blood is particularly preferred. Examples of blood include, but are not limited to, peripheral blood. When used in the method of the present invention, blood is preferably serum.

### Step (a)

For example, in blood, about 80% of PSA exists as a complex with α-antichymotrypsin (ACT). Therefore, in order to detect the target PSA with high sensitivity, in the step (a), PSA is released from the ACT-PSA complex present in the sample to generate free PSA (fPSA). Methods for releasing PSA is not particularly limited as long as it can release PSA from the ACT-PSA complex, but it is preferable to add a reagent to release it for ease of operation. The reagent used is not particularly limited as long as it can release PSA and does not interfere with subsequent PSA detection, but ethanolamine is preferably used. The conditions for the ethanolamine treatment are not particularly limited as long as they cleave the ACT-PSA complex and generate a sufficient amount of free PSA for detection, and for example, a method of treating at 37 to 42° C., preferably about 40° C., for 15 hours or more, preferably about 24 hours can be mentioned. By treatment at about 37° C. for about 24 hours, most of the ACT-PSA can be cleaved and free PSA can be produced.

When using blood (for example, serum) as a sample, it is preferable to perform a pretreatment of the sample before the step (a). The pretreatment includes, but is not limited to, affinity column, PSA immunoprecipitation, and various albumin/IgG removal methods. For albumin/IgG removal, various methods are reported and various devices and kits are on the market, and these can be used as appropriate. Albumin and IgG account for 60% or more of the proteins in human serum, so by removing them, the separation accuracy in two-dimensional electrophoresis in the step (b) can be improved.

In addition, in the method of the present invention, it is preferable that after treating PSA-ACT with ethanolamine to form free PSA in the step (a), serum is desalted by ultrafiltration and further concentrated by ultrafiltration and lyophilization.

The sample derived from the subject is preferably human-derived serum that has been subjected to albumin/IgG removal treatment and then ethanolamine treatment, and more preferably human-derived serum that has been further subjected to desalting by ultrafiltration and concentration by ultrafiltration and lyophilization.

### Step (b)

PSA in the sample is detected by performing two-dimensional electrophoresis on the free PSA (fPSA) obtained in the step (a) (including the free PSA originally present in the sample and the free PSA released from the complex in the step (a)) to separate PSA. In two-dimensional electrophoresis, for example, separation is performed based on isoelectric point in the 1st dimension and based on molecular weight in the 2nd dimension. This allows PSA in the sample to be separated based on isoelectric point and molecular weight. Since multiple types of PSA with different isoelectric points and molecular weights are usually present in a sample, the fPSA obtained in the step (a) can be detected as multiple spots the in step (b). Although not limited thereto, for example, five or more types of PSA spots, and depending on the detection sensitivity, eight or more types of PSA spots can be detected. Two-dimensional electrophoresis can be performed using a commercially available electrophoresis device and reagents, but high reproducibility can be obtained preferably by performing it using Auto 2D developed by the present inventors. Each separated PSA can be detected using a known method as appropriate. For example, but not limited to this, detection can be performed using Western blotting. Western blotting and detection conditions can be performed with appropriate reference to known techniques. It is convenient and preferable to use an antibody against PSA for detection.

### Step (c)

Step (c) is a step of verifying the presence of the PSA spot(s) detected on the basic side compared to the control PSA spot(s). When the PSA spot(s) detected in the step (b) is(are) compared with spot(s) of the control PSA(s) and the internal standard (fluorescent) labeled protein added during electrophoresis, and the detected spot(s) is(are) on the basic side than control, it is determined that more basic spot(s) exist(s) compared to the control. The case where the detected spot is more basic compared to the control includes, but not limited to, for example, cases wherein it has been verified that a PSA with a basic isoelectric point that is not detected in the control PSA is detected; that the isoelectric point of PSA which is the most abundant among PSAs with various isoelectric points is basic compared to normal PSA; that there are more fractions with a basic isoelectric point than in normal PSA; that when comparing the distribution of multiple spots, the spot distribution is on the basic side (shifted to the basic side) compared to the control; and the like. The case where there are more fractions with a basic isoelectric point than in normal PSA includes, for example, cases wherein it has been verified that based on a specific isoelectric point (examples thereof include, but not limited to, isoelectric points 5.8, 5.9, 6.0, 6.1, 6.2, 6.3, 6.4, or 6.5, preferably about 6) (hereinafter referred to as “reference isoelectric point”), the amount of PSA with an isoelectric point higher than the reference isoelectric point is greater than that of the control PSA; that comparing the amount of PSA with a reference isoelectric point or higher (hereinafter referred to as “basic side PSA”) with the amount of PSA with an isoelectric point less than the reference isoelectric point (hereinafter referred to as “acidic side PSA”), the ratio of basic side PSA to acidic side PSA is larger compared to control PSA; and the like. Any judgment method can be appropriately selected according to the actual circumstances of the measurement, such as measurement accuracy and purpose of measurement. For example, when the spot detected from the sample is on the basic side compared to the control PSA, by checking any of the above phenomena, then, it is judged or determined that a PSA spot exists on the basic side (or its existence is verified).

The control may be PSA derived from prostatic fluid of a healthy person, preferably free PSA prepared by pre-treatment, which usually results in multiple spots. A mixture of proteins with various isoelectric points and molecular weights prepared so as to produce spots similar to those of PSA derived from prostatic fluid of healthy persons (sometimes referred to as “control spots”) can also be used as the control. Furthermore, if the electrophoresis conditions are constant, a control spot may be determined in advance and the measurement target may be compared with it.

### Step (d)

In the step (d), when a PSA spot derived from the sample is detected on the more basic side than the control spot in the step (c), it is determined that the subject from which the sample is derived is suffering from or is suspected of suffering from prostate cancer. Furthermore, in the step (d), depending on the degree of PSA detected on the more basic side, the prostate cancer can be determined to be castration-resistant (hormone treatment-resistant) prostate cancer or suspected thereof. Detected on the more basic side includes, for example, that PSA with a basic isoelectric point not detected in PSA derived from hormone-sensitive prostate cancer patients (e.g., PSA with an isoelectric point of 6.5, 6.6, 6.7, 6.8, 6.9 or 7.0 or higher) is detected; that there are more fractions with more basic isoelectric points, that the distribution of the isoelectric point of PSA is on more basic side; and the like. More fractions with more basic isoelectric points include, for example, a case wherein when a reference isoelectric point is set and the amount of PSA with an isoelectric point equal to or higher than the reference isoelectric point is more than half of the total PSA; and the like. The reference isoelectric point referred to here can be, for example, 6.5, 6.6, or 6.7.

As described above, the present invention, which utilizes highly sensitive and highly specific markers, makes it possible to detect whether or not a subject has prostate cancer using the serum of test subject, and further to find a castration-resistant patient using the serum of a prostate cancer patient. The method of the present invention provides a highly specific and sensitive method for detecting prostate cancer using a simple method centered on sample pretreatment, separation, and detection. The method of the present invention is also a quantitative and highly accurate analysis method for post-translationally modified molecules of PSA, mainly based on a pretreatment method centered on specific separation and concentration of PSA complexes in serum, and a method for separating by isoelectric points and molecular weights (two-dimensional electrophoresis, etc.).

One of the most preferred embodiments of the present invention is a method for detecting prostate cancer, comprising the following steps; (i) a step of performing albumin/IgG removal treatment on a human-derived serum sample; (ii) a step of performing ethanolamine treatment on the albumin/IgG-removed serum, to release PSA from the PSA-ACT complex; (iii) a step of performing two-dimensional electrophoresis (1st dimension: isoelectric point, 2nd dimension: molecular weight) on the obtained free PSA using Auto 2D, for separating PSA, to detect the PSA spot; (iv) a step of verifying the presence of PSA detected on the basic side compared with control PSA, and (v) a step of determining that the subject (human) from which the serum is derived is suffering from prostate cancer when PSA is detected on the basic side in step (iv).

One of the most preferred embodiments of the present invention is a method for detecting castration-resistant prostate cancer, comprising determining that a subject has castration-resistant prostate cancer by verifying the presence of PSA detected on the more basic side using the above method. For example, the method of the present invention detects six or more PSA spots of 35 KD separated by isoelectric points, but those with the higher quantitative value of spots separated to the most basic side can be judged to be castration resistant. Therefore, these PSA spot groups will be quantified, and their distribution and profile will serve as a marker for castration resistance in prostate cancer.

Another aspect of the present invention is a method for determining prostate cancer, characterized by detecting PSA whose isoelectric point is basic compared to PSA present in prostatic fluid derived from a healthy person (normal PSA). In the determination method of the present invention, PSA present in a sample derived from a subject is detected, and when the detected PSA has an isoelectric point on the basic side compared to normal PSA, it is determined that the subject is suffering from prostate cancer. Furthermore, when the isoelectric point of the detected PSA is on the more basic side, it can be determined to be castration resistant.

Therefore, PSA having an isoelectric point on the more basic side than that of normal PSA is quantified, and its distribution and profile will serve as a marker for prostate cancer, and even serve as a marker for castration resistance in prostate cancer. Therefore, the present invention is a marker for prostate cancer, and further, a marker also for castration resistance of prostate cancer.

The method for measuring the isoelectric point of PSA in a sample is not limited, but it can be measured using, for example, two-dimensional electrophoresis described above. Alternatively, PSA in a sample can be roughly purified using a conventional method, and then measured using separation/analysis techniques based on the isoelectric point such as isoelectric focusing and the like.

Another aspect of the present invention is a method in which PSA present in a sample derived from a subject is detected and the isoelectric point of PSA is verified, and when PSA with an isoelectric point equal to or higher than a specific isoelectric point is detected, it is determined that the subject is suffering from prostate cancer. The subject-derived samples are the same as above.

The specific isoelectric point used for determining prostate cancer can be, for example, 6.0, 6.1, 6.2, 6.3, 6.4, or 6.5, and preferably, 6.0, 6.1 or 6.2, more preferably 6.0. When a PSA having an isoelectric point equal to or higher than a specific isoelectric point is detected, preferably when the detected value is high, it can be determined to be prostate cancer. Further, when a PSA having an isoelectric point equal to or higher than a specific isoelectric point, for example, equal to or higher than 6.5 is detected, preferably when the detected value is high, it can be determined that the subject has castration-resistant prostate cancer. Verification of the presence of PSA having the isoelectric point or higher in the sample can be performed using, for example, two-dimensional electrophoresis, although it is not limited thereto. Alternatively, PSA in a sample is separated using a conventional method, for example, using an antibody affinity column, and then a separation/analysis method based on the isoelectric point such as isoelectric focusing and the like is used, thereby the presence of PSA with the isoelectric point or higher can be verified.

Another aspect of the present invention is a method in which PSA present in a sample derived from a subject is detected and the degree of desialylation of PSA is detected, and when desialylated PSA is detected, it is determined that the subject is suffering from prostate cancer. Furthermore, if the degree of desialylated PSA is high, it can be determined that the subject has castration-resistant prostate cancer. Subject-derived samples are the same as above.

Verification of whether PSA in a sample has been desialylated can be performed using, for example, two-dimensional electrophoresis described above, although it is not limited thereto. When PSA is desialylated, the isoelectric point shifts, so by verifying this, the presence of desialylated PSA can be verified.

Therefore, the case where desialylated PSA is detected includes, for example, but not limited to, cases wherein it has been verified that PSA with an isoelectric point shifted due to desialylation (PSA with an isoelectric point that is not detected in normal PSA) is detected; that PSA with an isoelectric point that is expected to be detected in a normal PSA is not detected or is detected with a reduced number; and the like. The case where the degree of desialylated PSA is high includes, for example, cases in which a large amount of PSA with a specific shifted isoelectric point is verified; or when the amount of total PSA protein and sialic acid is quantified and the amount of sialylation per protein is determined and compared, the value is below a certain value; and the like.

Another aspect of the present invention is a method in which when prostate cancer or castration-resistant prostate cancer is determined by any of the above methods, the subsequent treatment policy will be determined based on that. That is, the present invention is also a method for determining prostate cancer for determining a treatment policy. The treatment policy is not limited, but for example, if it is diagnosed as prostate cancer but is not castration-resistant, that is, it is hormone-sensitive, hormone treatment is performed, while if it is determined to be castration-resistant prostate cancer, chemotherapy is selected.

Another aspect of the present invention is a method for evaluating the effectiveness of medication by detecting prostate-specific antigen (PSA) present in serum derived from prostate cancer patients receiving medication by any of the methods described above and comparing the detected PSA before and after medication.

Another aspect of the present invention is to evaluate the efficacy of a candidate therapeutic agent for prostate cancer using any of the methods described above. This is a method for evaluating or screening a new therapeutic drug.

### EXAMPLES

Hereinafter, the present invention will be specifically explained with reference to examples, but the present invention is not limited to the following examples.

### 1. Material

The followings were used as samples containing prostate-specific antigen (PSA): (i) seminal plasma derived from a healthy person, (ii) LNCaP cells (prostate cancer cells, PSA positive, sensitive to hormone therapy), (iii) C4-2 cells (prostate cancer cells, PSA positive, resistant to hormone therapy). As a control, PC-3 cells (prostate cancer cells, PSA negative) were used. In addition, the following patient sera were obtained after receiving certification from the Kumamoto University Ethics Review Committee and confirming informed consent, and used in the test: (a) hormone therapy-sensitive prostate cancer patient serum, (b) castration-resistant prostate cancer (CRPC) patient serum.

### (Example 1) Detection of PSA by Two-Dimensional Electrophoresis Using Healthy Person-Derived Seminal Plasma

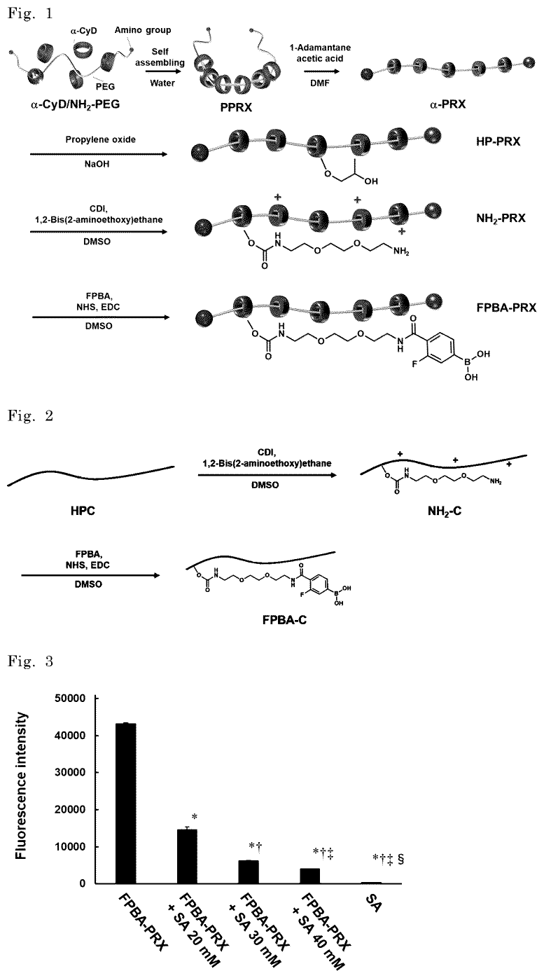

Seminal plasma (protein amount 10 μg) derived from a healthy person was applied to a fully automatic two-dimensional electrophoresis device (Auto 2D, manufactured by Sharp Manufacturing System Co., Ltd.) and two-dimensional electrophoresis and Western blotting were performed according to the manufacturer's instructions. The electrophoresis conditions are as follows. 1st dimension: pH 3-10, IPG gel, isoelectric focusing (IEF): 90 minutes; 2nd dimension: 10% polyacrylamide gel, SDS-PAGE: up to 40 minutes. After Western blotting, PSA spots were detected (primary antibody: polyclonal rabbit anti-human PSA antibody (Dako) or monoclonal mouse anti-human PSA antibody (R&D), secondary antibody: Cy5-goat-anti-Rabbit IgG, or Cy5-goat-anti-mouse IgG). Further, as internal 2D markers, Cy2 labeled trypsin inhibitor complex (PI: 4.5, M.W.: 83 KDa) and Carbonic anhydrase (PI: 4.5, M.W.: 83 KDa) were used. As a result, 17 or more PSA-positive spots that reacted with the above two antibodies were detected. The results are shown in FIG. 1. In the figure, eight representative spots are circled. As a result of performing LC-MS/MS analysis on each spot, PSA could be detected from five spots. Spots indicated by 2, 3, 6, 7, and 8 in the figure are spots where the presence of PSA was verified by LC-MS/MS analysis.

### (Example 2) Detection of PSA from LNCap Cells by Two-Dimensional Electrophoresis

In the same manner as in Example 1, PSA was detected using prostate cancer cells (LNCaP). A cell lysate was prepared from LNCaP cells, which are prostate cancer cells sensitive to hormone therapy, and two-dimensional electrophoresis was performed in the same manner as in Example 1 using 10 μg of the cell lysate in terms of protein amount. After Western blotting, a polyclonal anti-PSA antibody was used as the primary antibody. The results are shown in FIG. 2. Eight or more spots reactive with the anti-PSA antibody were verified.

### (Example 3) Detection of PSA by Two-Dimensional Electrophoresis Using Various Samples

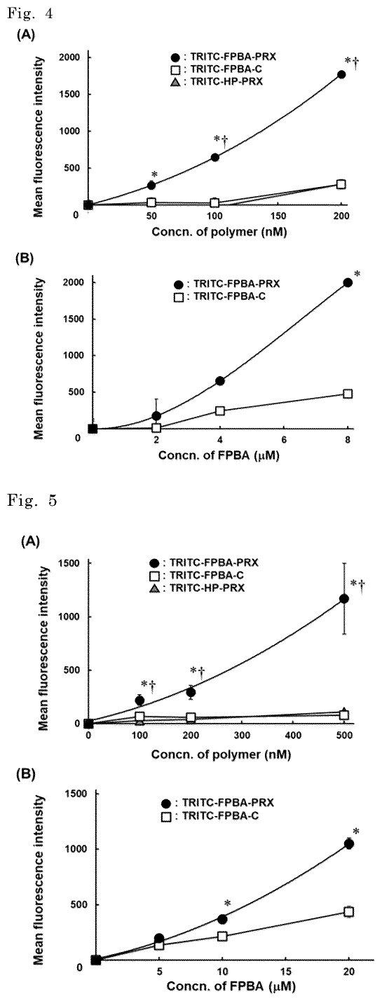

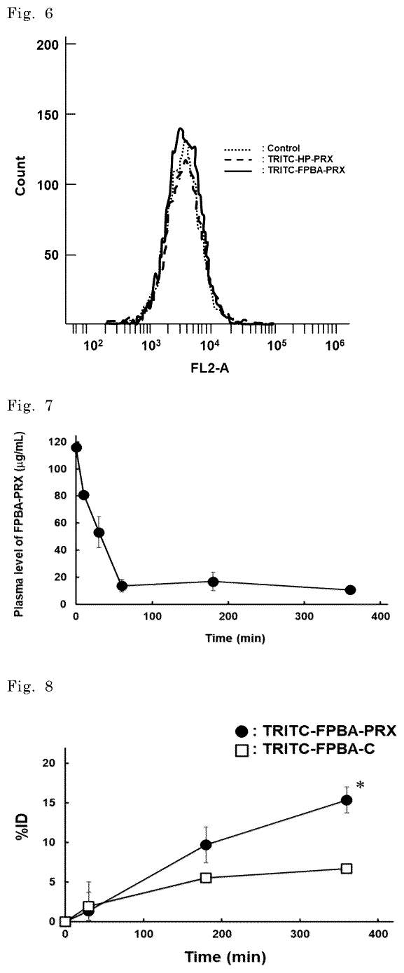

In the same manner as in Example 1, two-dimensional electrophoresis was performed on seminal plasma derived from a healthy person, a cell lysate of LNCaP cells, a cell lysate of C4-2 cells, and a cell lysate of PC-3 cells, and after Western blotting, PSA spots were detected using an anti-PSA antibody. The results are shown in FIG. 3. As a result, when comparing PSA spots (distribution) derived from healthy person seminal plasma and PSA spots derived from prostate cancer cells, it was verified that PSA spots (distribution) derived from prostate cancer cells (LNCaP cells and C4-2 cells) shifted to the basic side. Additionally, the shift to the basic side was more pronounced in C4-2, a prostate cancer cell resistant to hormone therapy.

### (Example 4) Measurement of Molecular Weight of PSA Present in Patient Serum

Patient serum was divided into fractions with molecular weights of 300 kDa or more, 100-300 kDa, 50-100 kDa, and 50 kDa or less using a filter.

Each fraction was treated in the same manner as in Example 1, except that a step of desalting by ultrafiltration and concentration by freeze-drying was added to the method before electrophoresis, and then two-dimensional electrophoresis was performed to detect PSA. Desalination by ultrafiltration was performed by washing with pure water, which was freeze-dried, redissolved in a trace amount of pure water of about 20 microliters, and concentrated. As a result, multiple spots that reacted with the same anti-PSA antibody were verified in the 300 kDa or more, 100-300 kDa, and 50 kDa or less fractions. The ACT-PSA complex present in serum is usually electrophoresed at around 100 kDa, but in reality it is thought to be 200-300 kDa or more in size. On the other hand, free PSA was verified in the fraction of 50 kDa or less.

It was found that when a protein of 100 kDa or more was fractionated and then treated with ethanolamine, a spot of free PSA appeared around 30 kDa.

Based on these facts, it was decided that PSA detection by two-dimensional electrophoresis using patient samples is performed in the presence of a reducing agent and a protein denaturing agent (urea) in isoelectric focusing, and in SDS electrophoresis in the 2nd dimension, a reducing agent and SDS (surfactant) are treated, in the following procedure.

### (Example 5) Detection of PSA by Two-Dimensional Electrophoresis Using Patient Sample (1)

PSA contained in the serum was detected using serum derived from prostate cancer patients. As a control, seminal plasma derived from a healthy person was used. Since the PSA concentration in blood is only on the order of ng per 1 mL, albumin and IgG were removed by pretreatment of the serum. The removal was performed using GE's albumin/IgG removal kit according to the manufacturer's instructions.

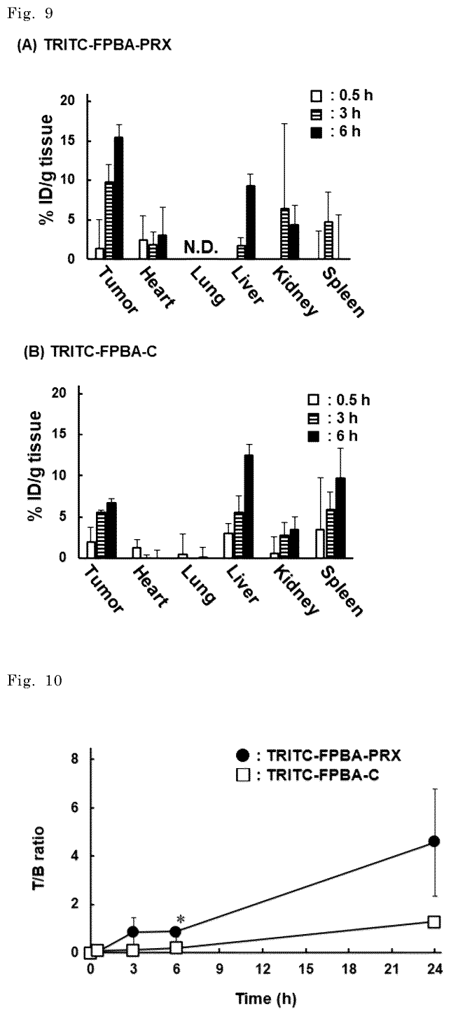

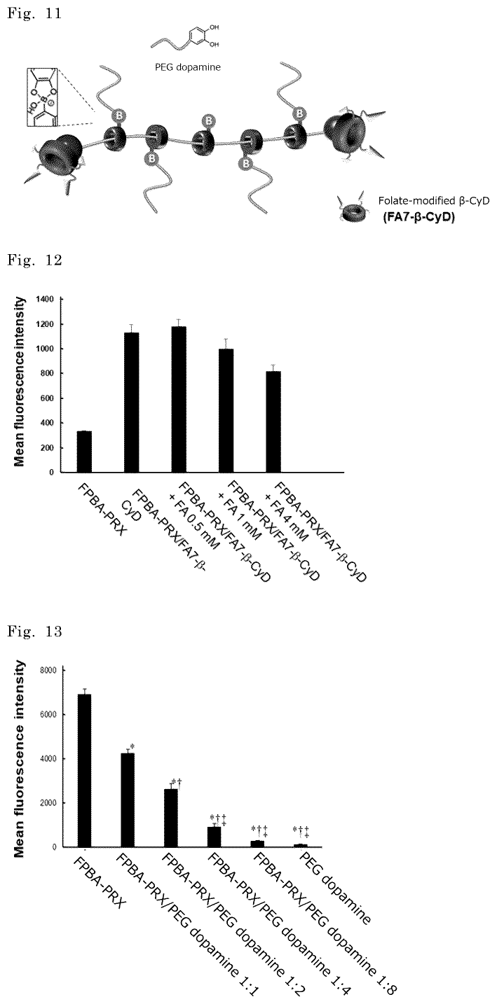

Next, ethanolamine treatment (0.1 M, 40° C., 24 hours) was performed, and after neutralization with hydrochloric acid, desalination by ultrafiltration, and concentration by freeze-drying, two-dimensional electrolysis was performed in the same manner as in Example 1 to detect PSA. The results are shown in FIG. 4. Furthermore, comparisons of detected spot intensities are shown in FIG. 5. As a result, it was verified that the intensity of spots on the basic side tended to be higher in serum derived from castration-resistant patients than in serum derived from hormone therapy sensitive patients. For example, although the evaluation method is not limited to this, when calculating the ratio of the sum of the intensities of spots 1 to 3 (A) to the sum of the intensities of spots 4 to 5 (B), A>B in serum derived from hormone therapy sensitive patients, whereas A≈B or A<B in serum derived from castration resistant patients. On the other hand, in the healthy person seminal plasma, the intensity of spots on the acidic side (1 to 3) was even higher. This indicates that the proportion of PSA on the basic side increases as castration resistance increases.

### (Example 6) Change in Isoelectric Point of PSA Due to Sialidase Treatment

PSA has undergone post-translational sugar chain modification and is known to have sialic acid, which is an acidic sugar. Then, the influence of sialidase was checked using two-dimensional electrophoresis using seminal plasma from a healthy person and serum from high PSA prostate cancer patients sensitive to hormone therapy.

Seminal plasma from a healthy person and serum from prostate cancer patients from which albumin/IgG had been removed were treated with sialidase, desalted by ultrafiltration, concentrated by freeze-drying, and then two-dimensional electrophoresis was performed in the same manner as in Example 1 to detect PSA.

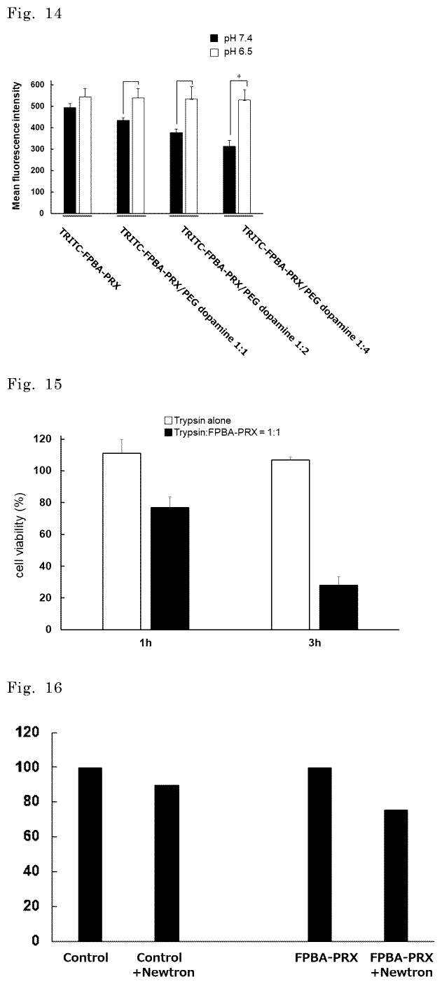

The results are shown in FIG. 6. It was verified that PSA was shifted to the basic side by sialidase treatment in all samples used. In addition, it was verified that some sugar chains are missing in some PSAs derived from prostate cancer patients, that is, partially desialylated. Furthermore, when PSA derived from castration-resistant patients was similarly investigated, it was verified that the proportion of PSA from which sugar chains have been removed increases as the patient becomes castration-resistant. Until now, there have been reports that sialidase activity is increased in prostate cancer compared to normal prostate cells, and that this tendency becomes stronger as they become castration-resistant, and the present results seem to reflect the deglycosylation of PSA due to this increase in sialidase.

### (Example 7) Detection of PSA by Two-Dimensional Electrophoresis Using Patient Sample (2)

Two-dimensional electrophoresis was performed in the same manner as in Example 5 using four examples for each of serum derived from hormone therapy-sensitive prostate cancer patients and serum derived from castration-resistant prostate cancer patients, respectively, to detect PSA. Thereafter, the detected spot intensities were compared. Specifically, the ratio of the sum of the intensities of the detected spots 1 to 3 (A) to the sum of the intensities of the 4 to 5 spots (B) was determined. The results are shown in FIG. 7. In serum derived from hormone therapy sensitive patients, A>B and the average ratio of A to B was approximately 1.7, whereas in serum derived from castration-resistant patients, A<B and the average ratio of A to B was approximately 0.7. It was verified that the number of basic PSA spots clearly increased in castration-resistant patients.

The above detailed description is merely illustrative of the purpose and subject matter of the present invention and is not intended to limit the scope of the appended claims. Various modifications and substitutions to the described embodiments will be apparent to those skilled in the art from the teachings described in the present specification without departing from the scope of the appended claims.

### INDUSTRIAL APPLICABILITY

The present invention is useful as a method for detecting prostate cancer based on a new marker. The present invention is also useful as a method for estimating the sensitivity of conventional prostate cancer treatment (hormone treatment) and determining the direction of treatment for a patient at an early stage, or is useful as a method for determining the effect of new therapeutic agents for prostate cancer.

1. A method for determining that a subject is suffering from or is suspected of suffering from prostate cancer by detecting prostate-specific antigen (PSA) present in a sample derived from the subject, characterized in that when the isoelectric point(s) of PSA(s) derived from the subject is on the basic side compared to PSA(s) derived from a healthy person, it is determined that the subject is suffering from or is suspected of suffering from prostate cancer.

2. The method according to claim 1, wherein when the isoelectric point of at least a portion of the PSA(s) derived from the subject is more basic than the isoelectric point of any PSA derived from a healthy person, when the fraction of PSA(s) that has basic isoelectric point derived from the subject is larger than the fraction of PSA(s) that has basic isoelectric point derived from a healthy person, when the distribution of the isoelectric point(s) of the PSA(s) derived from the subject is on the basic side compared to the distribution of the isoelectric point(s) of the PSA(s) derived from a healthy person, or when a PSA with an isoelectric point of 6.0 or higher is detected, it is determined that the isoelectric point(s) of the PSA(s) derived from the subject is on the basic side compared to PSA(s) derived from a healthy person.

3. The method according to claim 1, wherein when the fraction of PSA(s) that has basic isoelectric point derived from the subject is larger than the fraction of PSA(s) that has basic isoelectric point derived from a healthy person, or when the distribution of the isoelectric point(s) of PSA(s) derived from the subject is on the basic side compared to the distribution of the isoelectric point(s) of PSA(s) derived from a healthy person, it is determined that the isoelectric point(s) of the PSA(s) derived from the subject is on the basic side compared to PSA(s) derived from a healthy person.

4. The method according to any one of claims 1 to 3, wherein the sample is serum derived from the subject.

5. The method according to any one of claims 1 to 3, wherein the sample is serum derived from the subject treated with ethanolamine.

6. The method according to any one of claims 1 to 3, wherein the subject is further determined to have castration-resistant prostate cancer based on the degree of basicity of the isoelectric point of PSA derived from the subject.

7. The method according to any one of claims 1 to 3, wherein when PSA with an isoelectric point of 6.5 or more is detected, the subject is further determined to have castration-resistant prostate cancer.

8. A method for determining that a subject is suffering from or is suspected of suffering from prostate cancer by detecting prostate-specific antigen (PSA) present in a sample derived from the subject, wherein when PSA(s) derived from the subject is(are) desialylated compared to PSA(s) derived from a healthy person, it is determined that the subject is suffering from prostate cancer.

9. The method according to claim 8, wherein the sample derived from the subject is serum derived from the subject.

10. The method according to claim 8, wherein the sample derived from the subject is a subject-derived serum treated with ethanolamine.

11. The method according to any one of claims 8 to 10, wherein the subject is further determined to have castration-resistant prostate cancer based on the degree of desialylation of PSA(s) derived from the subject.

12. A method for obtaining information used to diagnose that a subject patient is suffering from or is suspected of suffering from prostate cancer, wherein the method comprises detecting prostate-specific antigen (PSA) present in serum derived from the patient suspected of having prostate cancer, and determining whether the isoelectric point(s) of the detected PSA(s) derived from the subject patient is on the basic side compared to PSA(s) derived from a healthy person.

13. The method according to claim 12, wherein when the isoelectric point of at least a portion of PSA(s) derived from the subject is more basic than the isoelectric point of any PSA derived from a healthy person, when the fraction of PSA(s) that has basic isoelectric point derived from the subject is larger than the fraction of PSA(s) that has basic isoelectric point derived from a healthy person, when the distribution of the isoelectric point(s) of PSA(s) derived from the subject is on the basic side compared to the distribution of the isoelectric point(s) of PSA(s) derived from a healthy person, or when a PSA with an isoelectric point of 6.0 or higher is detected, it is determined that the isoelectric point(s) of PSA(s) derived from the subject is on the basic side compared to PSA(s) derived from a healthy person.

14. The method according to claim or 13, wherein the serum derived from the patient is a patient-derived serum treated with ethanolamine.

15. A method for detecting prostate-specific antigen (PSA) present in a sample derived from a subject, comprising the following steps of:
(a) releasing PSA from a PSA-ACT complex present in the sample derived from the subject;
(b) performing two-dimensional electrophoresis on the free PSA obtained in the step (a) to separate PSAs with different isoelectric points and detecting each PSA spot; and
(c) verifying the presence of the PSA spot(s) detected on the basic side compared to the control PSA spot (s).

16. The method according to claim 15, wherein the sample derived from the subject is a human-derived serum.

17. The method according to claim 16, further comprises performing albumin/IgG removal treatment on the serum before the step (a).

18. The method according to any one of claims 15 to 17, wherein the step (a) is carried out using ethanolamine.

19. The method for determining that a subject is suffering from prostate cancer when a PSA spot is detected on the basic side in the step (c) in claim 18.

20. The method according to claim 19, wherein it is determined whether the subject is further suffering from castration-resistant prostate cancer, depending on the extent of the PSA spot(s) detected on the basic side compared to the control PSA spot, in the step (c).
